(19) United States
(12) Patent Application Publication
(10) Pub. No.: US 2018/0185427 A1
(43) Pub. Date: Jul. 5, 2018

(54) CASSYTHA FILIFORMIS AND/OR CUSCUTA SANDWICHIANA ADMINISTERED ORALLY TO INDUCE SLEEP

(71) Applicant: Performance Labs PTE. LTD., Singapore (SG)

(72) Inventor: Daniel Gubler, Orem, UT (US)

(21) Appl. No.: 15/861,388

(22) Filed: Jan. 3, 2018

Related U.S. Application Data
(60) Provisional application No. 62/442,098, filed on Jan. 4, 2017.

(51) Int. Cl. A61K 36/28; A61K 36/57; A61K 9/00; A61P 25/20

(52) U.S. Cl. CPC A61K 36/28; A61P 25/20; A61K 9/0053; A61K 36/57

(57) ABSTRACT

The present invention includes a composition and composition comprising: an extract of Cassytha filiformis, an extract of Cuscuta sandwichiana, or both, adapted for oral administration and in an amount sufficient to induce sleep, improve sleep, or both.

### CROSS-REFERENCE TO RELATED APPLICATIONS

This application claims priority to U.S. Provisional Application Ser. No. 62/442,098, filed Jan. 4, 2017, the contents of which are incorporated by reference herein in its entirety.

### STATEMENT OF FEDERALLY FUNDED RESEARCH

None.

### TECHNICAL FIELD OF THE INVENTION

The present invention relates in general to the field of inducing sleep, and more particularly, to novel compositions and methods for inducing sleep with isolated extracts from Cassytha filiformis and/or Cuscuta sandwichiana.

### BACKGROUND OF THE INVENTION

Without limiting the scope of the invention, its background is described in connection with insomnia.

One such method of treating insomnia U.S. Pat. No. 9,408,997, issued to Kelley and entitled, “System and method for the treatment of insomnia”, which teaches a system and method that provides controlled bilateral stimulation to a patient in response to physiological feedback to initiate and/or accelerate the onset of sleep and to maintain the sleep state after the onset of sleep. The system induces bilateral stimulation with modules that are coupled to opposite lateral sides of the patient's body. The system controls the duration of the stimulations, the intensity of the stimulations and the period of time between successive stimulations in response to feedback from physiological sensors, which are coupled to the patient and provide physiological information, which initiates or terminates the bilateral stimulations, and/or modifies the characteristics of the stimulations.

Another such patent is U.S. Pat. No. 8,372,453, issued to Kim, et al., and entitled “Composition for prevention or treatment of insomnia”, and teaches a composition that includes, as an active ingredient, an extract of Phlomis umbrosa or a combination thereof with extract of Polygala tenuifolia Willd. Particularly, the combination of extract of Phlomis umbrosa with extract of Polygala tenuifolia that enables recovery of the sleep of patients suffering from insomnia nearly to that of normal persons. The composition for treating insomnia uses active ingredients that have been used as Chinese traditional medicines, and thus is safe to the human body.

Another such patent is U.S. Pat. No. 8,252,290, issued to Chan and entitled “Herbal composition for treatment of insomnia and other related disorders and a method of preparing the same”, which teaches an herbal composition that comprises an extract of Radix Rehmanniae, and an extract of Poria, Ganoderma Lucidum, Semen Ziziphi Spinosae, Semen Platycladi, Radix Rehmanniae Praeparata, Radix Angelicae Sinensis, Radix Et Rhizoma Salviae Miltiorrhizae, Rhizoma Chuanxiong, Rhizoma Atractylodis Macrocephalae, Fructus Gardeniae, Cortex Moutan, Radix Bupleuri, Radix Paeoniae Alba, Rhizoma Cyperi, Pericarpium Citri Reticulatae, Fructus Aurantii, Radix Glycyrrhizae, and Os Draconis. The herbal composition is prepared by separately extracting Radix Rehmanniae, and the remaining herbs. The composition is suitable for treatment of insomnia and other related disorders.

Yet another example is U.S. Patent Application Publication No. 2013/0230589, filed by Pettersson, et al., and entitled “Pharmaceutical Formulations Useful in the Treatment of Insomnia”, is said to teach a formulation suitable for transmucosal administration comprising a short acting hypnotic drug, which formulation provides a measurable plasma concentration of drug within 10 minutes of administration. The formulation is said to be capable of providing sleep on demand, and preferably comprises particles of drug, for example zolpidem or a pharmaceutically-acceptable salt thereof and a mucoadhesion promoting agent, such as sodium carboxymethylcellulose, which particles of drug and mucoadhesive are presented upon the surface of larger carrier particles.

### SUMMARY OF THE INVENTION

In one embodiment, the present invention includes a composition comprising: an extract of Cassytha filiformis, an extract of Cuscuta sandwichiana, or both, adapted for oral administration and in an amount sufficient to induce sleep, improve sleep, or both. In one aspect, the extracts of Cassytha filiformis and Cuscuta sandwichiana are provided in an amount that is synergistic to induce sleep, improve sleep, or both. In another aspect, the extract of Cassytha filiformis, Cuscuta sandwichiana, or both, are provided in a receptacle that is water-permeable and withstands boiling water. In another aspect, the extract of Cassytha filiformis, Cuscuta sandwichiana, or both, are in a tea bag, capsule, caplet, softgel, gelcap, suppository, film, granule, pellet, tablet, mini-tablet, enveloped, in or on a bead, troche, lozenge, disk, poultice, wafer, solution, suspension, cream, ointment, lotion, enema, elixir, syrup, emulsion, gum, gummy, insert, jelly, foam, paste, pastille, spray, magma or poultice. In another aspect, the Cassytha filiformis, Cuscuta sandwichiana, or both, are provided for immediate release, extended release, or both. In another aspect, the composition further comprises an excipient selected from the group consisting of sweetening agents, colorants, surfactants, flavors, fragrances, pH modifiers, bulking agents, and mixtures thereof. In another aspect, the extract of Cassytha filiformis is provided in an amount of 1.2 grams to 1.8 grams per dose. In another aspect, the extract of Cuscuta sandwichiana is provided in an amount of 0.2 grams to 0.8 grams per dose. In another aspect, both the Cassytha filiformis and Cuscuta sandwichiana are provided in an amount of 2.0 grams to 3.0 grams per dose. In another aspect, a whole plant powder of Cassytha filiformis, Cuscuta sandwichiana, or both is used. In another aspect, the composition is a whole plant powder of Cassytha filiformis, and the composition further comprises a source of Vitamin E. In another aspect, the composition is extracted with hot water. In another aspect, the composition consists essentially of between 2.0 grams to 3.0 grams per day of the water extract of Cassytha filiformis, Cuscuta sandwichiana, or both. In another aspect, the composition consists of between 2.0 grams to 3.0 grams per day of an extract of Cassytha filiformis, Cuscuta sandwichiana, or both.

In another embodiment, the present invention includes a method for inducing or improving sleep in a patient in need thereof consisting of administering to the patient a plant composition, the plant composition comprising a water extract of Cassytha filiformis, Cuscuta sandwichiana, or both, adapted for oral administration and in an amount sufficient to induce or improve sleep. In one aspect, the extracts of Cassytha filiformis and Cuscuta sandwichiana are provided in an amount that is synergistic to induce or improve sleep. In another aspect, the extract of Cassytha filiformis, Cuscuta sandwichiana, or both, are provided in a receptacle that is water-permeable and withstands boiling water. In another aspect, the extract of Cassytha filiformis, Cuscuta sandwichiana, or both, are contained in a tea bag. In another aspect, the extract of Cassytha filiformis, Cuscuta sandwichiana, or both, are in a tea bag, capsule, caplet, softgel, gelcap, suppository, film, granule, pellet, tablet, mini-tablet, enveloped, in or on a bead, troche, lozenge, disk, poultice, wafer, solution, suspension, cream, ointment, lotion, enema, elixir, syrup, emulsion, gum, gummy, insert, jelly, foam, paste, pastille, spray, magma or poultice. In another aspect, the Cassytha filiformis, Cuscuta sandwichiana, or both, are provided for immediate release, extended release, or both. In another aspect, the method further comprises an excipient selected from the group consisting of sweetening agents, colorants, surfactants, flavors, fragrances, pH modifiers, bulking agents, and mixtures thereof. In another aspect, the extract of Cassytha filiformis is provided in an amount of 1.2 grams to 1.8 grams per dose. In another aspect, the extract of Cuscuta sandwichiana is provided in an amount of 0.2 grams to 0.8 grams per dose. In another aspect, the Cassytha filiformis is provided in an amount of 1.2 grams to 1.8 grams per dose and the Cuscuta sandwichiana is provided in an amount of 0.2 grams to 0.8 grams per dose. In another aspect, the Cassytha filiformis, Cuscuta sandwichiana, or both, are provided at least 30 minutes before sleep. In another aspect, the method further comprises the step of identifying a subject in need of improved sleep prior to providing the composition. In another aspect, a whole plant powder of Cassytha filiformis, Cuscuta sandwichiana, or both is used. In another aspect, the composition is a whole plant powder of Cassytha filiformis, and the composition further comprises a source of Vitamin E. In another aspect, the method further comprises the step of extracting the composition with hot water. In another aspect, the composition consists essentially of between 2.0 grams to 3.0 grams per day of the water extract of Cassytha filiformis, Cuscuta sandwichiana, or both. In another aspect, the composition consists of between 2.0 grams to 3.0 grams per day of the water extract of Cassytha filiformis, Cuscuta sandwichiana, or both.

Yet another embodiment of the present invention includes a method for evaluating the amount of an extract Cassytha filiformis, Cuscuta sandwichiana, or both, adapted for oral administration needed to induce or improve sleep, the method comprising: determining an average amount of time needed to reach a state of sleep in one or more subjects; providing an amount of the extract Cassytha filiformis, Cuscuta sandwichiana, or both, orally to a first subset of the subjects, and a placebo to a second subset of the subjects; determining an average amount of time needed to reach a state of sleep in the first subset of the subjects, and the second subset of the subjects; and determining if the amount of the extract Cassytha filiformis, Cuscuta sandwichiana, or both, induces sleep in a shorter time, improves a quality of sleep, or both that is statistically significant as compared to any reduction occurring in the second subset of patients, wherein a statistically significant reduction indicates that the candidate drug is useful in treating said disease state.

In another embodiment, the present invention includes a composition consisting essentially of an extract of Cassytha filiformis, an extract of Cuscuta sandwichiana, or both, adapted for extraction with hot water and oral administration in an amount sufficient to induce or improve sleep. In one aspect, the both the extracts of Cassytha filiformis and Cuscuta sandwichiana are provided in an amount that is synergistic for induce sleep, improve sleep, or both. In another aspect, the extract of Cassytha filiformis is provided in an amount of 1.2 grams to 1.8 grams per dose. In another aspect, the extract of Cuscuta sandwichiana is provided in an amount of 0.2 grams to 0.8 grams per dose. In another aspect, the Cassytha filiformis is provided in an amount of 1.2 grams to 1.8 grams per dose and the Cuscuta sandwichiana is provided in an amount of 0.2 grams to 0.8 grams per dose. In another aspect, the Cassytha filiformis, Cuscuta sandwichiana, or both, are provided at least 30 minutes before sleep.

In another embodiment, the present invention includes a composition consisting of an extract of Cassytha filiformis, an extract of Cuscuta sandwichiana, or both, adapted for extraction with hot water and oral administration in an amount sufficient to induce sleep. In one aspect, the both the extracts of Cassytha filiformis and Cuscuta sandwichiana are provided in an amount that is synergistic for induce sleep, improve sleep, or both. In another aspect, the extract of Cassytha filiformis is provided in an amount of 1.2 grams to 1.8 grams per dose. In another aspect, the extract of Cuscuta sandwichiana is provided in an amount of 0.2 grams to 0.8 grams per dose. In another aspect, the Cassytha filiformis is provided in an amount of 1.2 grams to 1.8 grams per dose and the Cuscuta sandwichiana is provided in an amount of 0.2 grams to 0.8 grams per dose. In another aspect, the Cassytha filiformis, Cuscuta sandwichiana, or both, are provided at least 30 minutes before sleep.

Yet another embodiment of the present invention includes a method for inducing or improving sleep in a patient in need thereof consisting essentially of administering to the patient a plant composition, the plant composition comprising an extract of Cassytha filiformis, Cuscuta sandwichiana, or both, adapted for oral administration and in an amount sufficient to induce or improve sleep.

Another embodiment of the present invention includes a method for inducing or improving sleep in a patient in need thereof consisting of administering to the patient a plant composition, the plant composition consisting of an extract of Cassytha filiformis, Cuscuta sandwichiana, or both, adapted for oral administration and in an amount sufficient to induce or improve sleep.

### BRIEF DESCRIPTION OF THE DRAWINGS

For a more complete understanding of the features and advantages of the present invention, reference is now made to the detailed description of the invention along with the accompanying figures and in which:

FIG. 1 shows the chemical Structures of Some Aporphine Alkaloids Found in C. filiformis.

FIG. 2 shows the chemical analysis using a Gas Chromatography-Mass Spectroscopy (GC-MS) analysis of a water extracted from a C. sandwichiana extract.

FIG. 3 shows the chemical structures of the active ingredients of the present invention, specifically, compounds water extracted from a C. sandwichiana extract.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

This invention details the ability of a tea comprising extracted aporphine alkaloids and/or Vitamin E obtained from whole plant powder of either Cassytha filiformis or Cuscuta sandwichiana, respectively, to induce sleep due to the presence of respectively. While each has a tradition of being used in vapor form to help relax a user, the present invention is based on the surprising effectiveness of an extract of either extract, or a combination of extracts.

As used herein, the term “Cassytha filifiormis” (C. filiformis) refers to a parasitic twining dodder that grows in tropical regions throughout the world—with reports of it growing in Nigeria, Taiwan, China, and the Hawaiian Islands in the United States. It is a leafless plant that requires a host plant to survive—seedlings of C. filiformis needing to find a host plant to attach to 8-10 days after germination or the plant will die. C. filiformis can vary in color from a dark green to a bright orange depending on the maturity of the plant.

Attachment of C. filiformis to its host plant is accomplished by the presence of suction-like cups or tentacles called haustoria that stick into the vascular system of the host plant from which derives the necessary water and nutrients for survival.

C. filiformis is not particular on the type of host plant it chooses, grows quite rapidly, and can overrun the host plant. It is considered an invasive weed in many parts of the world with pesticide being sprayed to keep the populations down.

C. filiformis has been used in in the traditional medicine regiment of several cultures for thousands of years. In Nigeria, C. filiformis was traditionally used to treat African sleeping sickness, diabetes, ulcers hemorrhoids, cough, as well as many forms of cancer. The seeds of C. filiformis are used in traditional Chinese medicine as a potent aphrodisiac. Toxicity studies of C. filiformis have been performed and have found this plant to possess no toxicity to humans at feasible concentrations.

As used herein, the term “Cuscuta Sandwichiana” (C. sandwichiana) refers to a parasitic twining dodder plant with a similar general appearance to C. filiformis. C. sandwichiana is endemic to the Hawaiian islands and, according to current scientific knowledge, is still only found in the Hawaiian islands. This plant is currently found in the islands in natural preserves such as Ka'ena Point—Oahu's Northernmost tip.

Although these two plants look similar there are some distinguishing features. The flowering pattern of C. sandwichiana is quite different from C. filiformis. However, it can be quite difficult to tell the two plants apart if they are not flowering. The main distinguishing feature to aid the identification of these two plants is the thickness of the vine.

The Hawaiians revered both C. filiformis and C. sandwichiana (called Kauna'oa Pehu—Pehu meaning “thick”—and Kauna'oa in Hawaiian respectively) as sacred plants given to them by the volcano goddess Pele—hence the orange lava-like color of the mature plants. The Hawaiians would eat both C. filiformis and C. sandwichiana as part of their diet with other green vegetables. Among the most interesting uses of C. filiformis and C. sandwichiana is found in traditional Hawaiian medicine to relax users. Hawaiian traditional medicine practitioners draw a hot water bath and to get the water as hot as the person can stand. After having the person sit in the hot water bath, fresh Kauna'oa (C. filiformis or C. sandwichiana) was added—causing the water to turn a yellow/orange color. According to the accounts of these practitioners, sitting in the hot water bath for about 10 minutes is enough to cause a peaceful and fitful sleep for those suffering from insomnia.

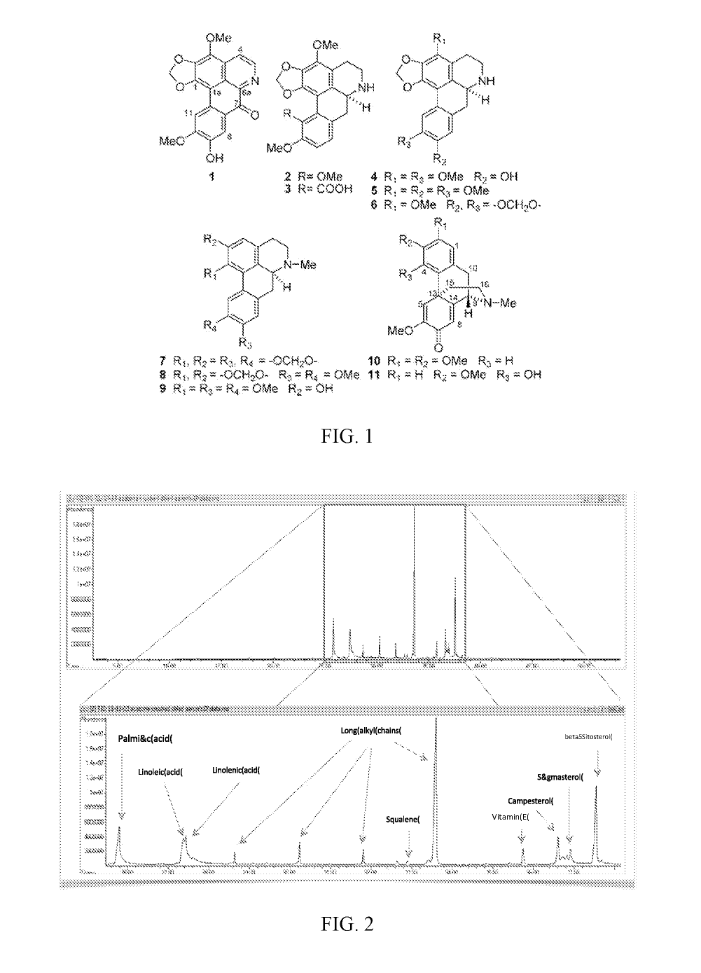

FIG. 1 shows the chemical Structures of Some Aporphine Alkaloids Found in C. filiformis. Adapted from Tung-Hu Tsai, Guei-Jane Wang, and Lie-Chwen Lin, “Vasorelaxing Alkaloids and Flavonoids from Cassytha filiformis”, J. Nat. Prod. 2008, 71, 289-291.

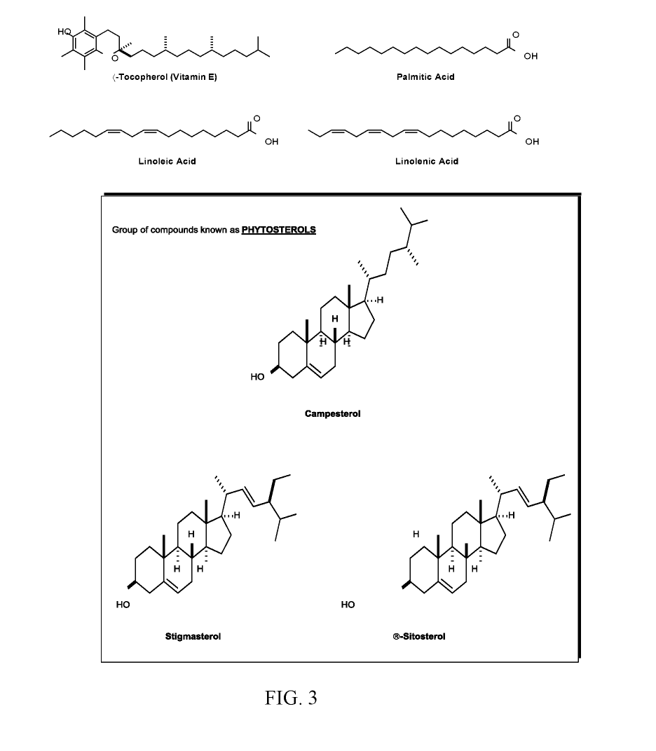

While the chemical composition of C. filiformis was known, the chemical composition of C. sandwichiana was not known. Gas Chromatography-Mass Spectroscopy (GC-MS) analysis conducted the present inventor (FIGS. 2 and 3 below) have shown that the major organic components of C. sandwichiana are vitamin E, palmitic acid, linoleic acid, linolenic acid, and plant sterols. In house studies showed that C. sandwichiana does not possess any toxicity in regards to human consumption.

The key innovation of this patent lies in the development of a tea using plant material from either C. filiformis and/or C. sandwichiana in order to help induce sleep. There is a great need on the world today for a natural-based sleep aid that does not interfere with the normal circadian rhythm and without side effect. Pharmaceutical and OTC sleep aids use synthetic molecules and are based on a monopharmacology (one molecule, one biological target) strategy. This nutraceutical novel approach of using either C. filiformis or C. sandwichiana is based on a polypharmacology approach—meaning several organic compounds in these plants bind to different sites in the body to cause a beneficial pharmacological effect that can't be matched by using a single molecule.

The premise for this idea was based on the oral traditional among Hawaiian traditional medicine healers that placing either of these plant materials in a hot water bath is effective at inducing a state of relaxation. This knowledge has been passed on orally. The mode of action of the traditional medicine healer combines increasing the temperature of the user via a hot water bath followed by inhaling the volatile organic plant compounds coming off of the hot water in an aromatherapy fashion. The other potential mode of action would be that the organic compounds in the water solution were being absorbed through the skin—which is not likely to happen due to the skin impenetrability of the organic compounds found in either plant.

With the sleep-inducing activity of the plants likely coming from an aromatherapy mode of action and, given absence of human toxicity for both plants, the present inventor reasoned that making a tea out of dried powder material could provide improved bioactivity versus the aromatherapy route—and likely even an enhanced effect due to the dual effects of both aromatherapy and actual ingestion of aporphine alkaloids into the gut. See below for the details on the preparation of the plant material and sleep tea.

Sleep Tea Preparation and Instructions: (1) Either C. filiformis and/or C. sandwichiana was carefully removed from the host plant so as to not cause damage or abrasion to either plant. Care was taken to ensure that neither C. filiformis nor C. sandwichiana was completed removed from the host plant so regeneration would occur. (2) The vine was carefully rinsed with distilled water and patted dry with paper towels. (3) Let air dry on a counter or open space for one hour or until dry. (4) Vine is placed in a brown paper bag and holes are poked in the bag for good ventilation. A cardboard box can also be used. (5) Vine in brown paper bag is placed in an arid place, at room temperature, out of the sun for to air dry for about 1 week. Drying time can be longer or shorter depending on the amount of material. The key indicator is that the vine will turn a dull brown color and will become quite brittle. If the vine is not brittle then it needs to be dried longer.

The dried brittle vine is then gently ground into a coarse powder either using a mortar and pestle or a commercial blender in pulse mode. In both cases care must be taken to make sure that the powder does not heat up—as this can cause decomposition of the bioactive constituents (aporphine alkaloids in C. filiformis and Vitamin E in C. sandwichiana) in the plants. Pour the finished powder into glass vials and store at room temperature away from direct sunlight. For long term storage the material is best kept at 4° C. This material is then used to prepare the sleep tea.

Sleep Tea Preparation and Instructions: (1) Weigh out 2.0 grams of the plant material (prepared as detailed above), put into a tea bag and seal the tea bag. Alternatively the plant material could be placed directly into a French Press if that method is preferred. (2) Add tea bag to 6 ounces of boiling water (212° C.) and let steep for 5-10 minutes. (3) Drink 30 minutes before retiring to bed.

Study: Study Design: Twenty subjects drank one tea bag of either C. filiformis or C. sandwichiana each night for 30 consecutive nights. The tea was steeped with boiling water for 5 minutes and then immediately consumed once the tea solution had cooled sufficiently.

Study Result: Shown below are the results from the study for both C. filiformis and C. sandwichiana.

C. filiformis Results:

Percentage of participants reporting better sleep quality: 97%
Percentage of participants waking up more refreshed: 90%

Participants taking C. filiformis reported having more vivid dreams—suggesting that more time was spent in REM sleep. REM sleep (in moderate amounts) has been associated with a good quality sleep, improved cognition, and better overall health in a myriad of different categories. The majority of participants also reported that they fell they were more rejuvenated and less stressed when waking up in the morning. The fact that the participants had REM sleep but woke up refreshed likely means they had an essential amount of REM sleep but also went into periods of deep sleep (stage 2-3).

C. sandwichiana Results:

Percentage of participants reporting better sleep quality: 73%
Percentage of participants waking up more refreshed: 70%

Proposed Mode of Action: By way of explanation, and in no way a limitation of the present invention, the present inventors hypothesize, based on the present studies, that it is likely that the presence of the aporphine alkaloids in C. filiformis may be responsible for the beneficial sleep properties. Aporphine alkaloids are privileged scaffolds in medicinal chemistry that have demonstrated a potent ability to inhibit both serotonin (h5-HT1A and h5-HT2A) and dopamine D3 receptors of the brain. Increased levels of serotonin and dopamine in the brain help to induce sleep and naturally supplement the body's normal circadian rhythm.

The study further demonstrated that C. sandwichiana is less effective than C. filiformis at inducing sleep. This is most certainly due to the fact that aporphine alkaloids are found in C. filiformis and are absent in C. sandwichiana. The sleep inducing properties of C. sandwichiana are likely due to the large amounts of vitamin E contained therein, which is believed to help those with restless leg syndrome (RLS), which is a major contributor to insomnia.

This invention describes the development of an oral, water extracted composition using C. filiformis and/or C. sandwichiana that has the ability to naturally induce sleep via a polypharmacology approach without the harmful side effects found in prescription sleep aids.

The chemical composition of C. filiformis has been studied in some detail and is reported in the peer-reviewed clinical literature. The most interesting secondary metabolites for in C. filiformis are aporphine alkaloids like those shown in FIG. 3. Aporphine alkaloids found in C. filiformis and other plants have been shown to have a wide range of biological activities. Among the most pertinent to this invention disclosure is that aporphine alkaloids have been found to be a “privileged scaffold” in their ability to inhibit both serotonin and dopamine receptors in the brain. When used together with the natural source of Vitamin E, the present invention provides a surprising and unexpected benefit to the users of the compositions.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Na Ye, QianQian Wu, Liyuan Zhu, Longtai Zheng, Bo Gao, Xuechu Zhen, Ao Zhang, “Further SAR study on 11-O-substituted aporphine analogues: Identification of highly potent dopamine D3 receptor ligands” Bioorganic & Medicinal Chemistry 19 (2011) 1999-2008.

Scot C. Nelson, “Cassytha filiformis” Cooperative Extension Service, University of Hawaii at Manos, Plant Disease, July 2008.

N. Armenia, Hercegovina, Dian Gustinanda, Annisa Nur Salasa, Yori Yuliandra and Friardi, “Acute And Delayed Toxicity Study Of Cassytha Filiformis Defatted Ethanolic Extract”, World Journal Of Pharmacy And Pharmaceutical Sciences, Volume 4, Issue 10, 155-162 (2015).

Avi Karni, David Tanne, Barton S. Rubenstein, Jean J. M. Askenasy and Dov Sagi, “Dependence on REM Sleep of Overnight Improvement of a Perceptual Skill”, Science, New Series, Vol. 265, No. 5172 (Jul. 29, 1994), pp. 679-682.

Nirav Kapadia, Wayne W. Harding, “C4 phenyl aporphines with selective h5-HT2B receptor affinity”, Bioorganic & Medicinal Chemistry Letters 25 (2015) 3451-3454.

Shashikanth Ponnala, Nirav Kapadia, Sudharshan Madapa, Ian L. Alberts, Wayne W. Harding, “Synthesis and evaluation of aporphine analogs containing C1 allyl isosteres at the h5-HT2A receptor”, Bioorganic & Medicinal Chemistry Letters 25 (2015) 5102-5106.

P. H. Kelly, R. J. Miller & J. L. Neumeyer, “Effect of aporphine alkaloids on central dopamine receptors”, Proceedings Of The B.P.S., 26-27 Mar. 1975, 271P.

Hausatu M. Babayi, B. Techa, Joseph, J. I. Udeme Prof, Joseph A. Abalaka Prof, Joseph I. Okogun Prof, O. A. Salawu, Ph.D, David D. Akumka MSc, Adamu, HND, Sunday S. Zarma, OND, Bulus B. Adzu MSc, Sabo S. Abdulmumuni B S, Kolo Ibrahime, Baba B. Elisha HND, Samuel S. Zakariys, HN, Uford S. Inyang, PhD, “Effect of Oral Administration of Aqueous Whole Extract of Cassytha Filiformis on Haematograms and Plasma Biochemical Parameters in Rats”, Journal Of Medical Toxicology, Volume 3, Number 4, December 2007, 146-151.

Fang-Rong Chang, Ya-Chieh Chao, Che-Ming Teng, and Yang-Chang Wu, “Chemical Constituents from Cassytha filiformis II”, Journal of Natural Products, Volume 61, Number 7, July 1998, 863-866.

Tung-Hu Tsai, Guei-Jane Wang, and Lie-Chwen Lin, “Vasorelaxing Alkaloids and Flavonoids from Cassytha filiformis”, J. Nat. Prod. 2008, 71, 289-291.

Yang-Chang Wu,* Ya-Chieh Chao, Fang-Rong Chang And Yuan-Yng Chen, “Alkaloids From Cassytha Filiformis”, Phytochemistry. Vol. 46, No. 1, pp. 181-184. 1997.

Caroline Stevigny1 Marie-Caroline Wautier1 Jean-Louis Habib Jiwan2 Patrice Chiap Philippe Hubert3 Joelle Quetin-Leclercq1, “Development and Validation of a High Performance Liquid Chromatographic Method for Quantitative Determination of Aporphine Alkaloids from Different Samples of Cassytha filiformis”, Planta Med 2004; 70: 764-770.

Mythili Sathiavelu, Sathiavelu Arunachalam, “High performance thin layer chromatography profile of Cassytha filiformis”, Asian Pacific Journal of Tropical Biomedicine (2012) S1431-S1435.

Mohammad Mahdi Sagheb, Banafshe Dormanesh, Mohammad Kazem Fallahzadeh, Hamideh Akbari, Sahar Sohrabi Nazari, Seyed Taghi Heydari, Saeed Behzadi, “Efficacy of vitamins C, E, and their combination for treatment of restless legs syndrome in hemodialysis patients: A randomized, double-blind, placebo-controlled trial”, Sleep Medicine 13 (2012) 542-545.

What is claimed is:

1. A composition comprising:
an extract of Cassytha filiformis, an extract of Cuscuta sandwichiana, or both, adapted for oral administration and in an amount sufficient to induce sleep, improve sleep, or both.

2. The composition of claim 1, wherein the extracts of Cassytha filiformis and Cuscuta sandwichiana are provided in an amount that is synergistic to induce sleep, improve sleep, or both.

3. The composition of claim 1, wherein the extract of Cassytha filiformis, Cuscuta sandwichiana, or both, are provided in a receptacle that is water-permeable and withstands boiling water, a tea bag, capsule, caplet, softgel, gelcap, suppository, film, granule, pellet, tablet, mini-tablet, enveloped, in or on a bead, troche, lozenge, disk, poultice, wafer, solution, suspension, cream, ointment, lotion, enema, elixir, syrup, emulsion, gum, gummy, insert, jelly, foam, paste, pastille, spray, magma or poultice.

4. The composition of claim 1, wherein the Cassytha filiformis, Cuscuta sandwichiana, or both, are provided for immediate release, extended release, or both.

5. The composition of claim 1, further comprising an excipient selected from the group consisting of sweetening agents, colorants, surfactants, flavors, fragrances, pH modifiers, bulking agents, and mixtures thereof.

6. The composition of claim 1, wherein the extract of Cassytha filiformis is provided in an amount of 1.2 grams to 1.8 grams per dose; or wherein the extract of Cuscuta sandwichiana is provided in an amount of 0.2 grams to 0.8 grams per dose; or wherein the combination of Cassytha filiformis and Cuscuta sandwichiana are provided in an amount of 2.0 grams to 3.0 grams per dose.

7. The composition of claim 1, wherein a whole plant powder of Cassytha filiformis, Cuscuta sandwichiana, or both is used.

8. The composition of claim 1, wherein the composition is a whole plant powder of Cassytha filiformis, and the composition further comprises a source of Vitamin E.

9. The composition of claim 1, wherein the composition is extracted with water that is sufficiently hot to extract aporphine alkaloids, or Vitamin E, respectively, or boiling water.

10. The composition of claim 1, wherein the composition consists essentially of between 2.0 grams to 3.0 grams per day of the water extract of Cassytha filiformis, Cuscuta sandwichiana, or both.

11. The composition of claim 1, wherein the composition consists of between 2.0 grams to 3.0 grams per day of an extract of Cassytha filiformis, Cuscuta sandwichiana, or both.

12. A method for inducing or improving sleep in a patient in need thereof comprising administering to the patient a plant composition, the plant composition comprising a water extract of Cassytha filiformis, Cuscuta sandwichiana, or both, adapted for oral administration and in an amount sufficient to induce or improve sleep.

13. The method of claim 12, wherein the extracts of Cassytha filiformis and Cuscuta sandwichiana are provided in an amount that is synergistic to induce or improve sleep.

14. The method of claim 12, wherein the extract of Cassytha filiformis, Cuscuta sandwichiana, or both, are provided in a receptacle that is water-permeable and withstands boiling water, a tea bag, capsule, caplet, softgel, gelcap, suppository, film, granule, pellet, tablet, mini-tablet, enveloped, in or on a bead, troche, lozenge, disk, poultice, wafer, solution, suspension, cream, ointment, lotion, enema, elixir, syrup, emulsion, gum, gummy, insert, jelly, foam, paste, pastille, spray, magma or poultice.

15. The method of claim 12, wherein the Cassytha filiformis, Cuscuta sandwichiana, or both, are provided for immediate release, extended release, or both.

16. The method of claim 12, further comprising an excipient selected from the group consisting of sweetening agents, colorants, surfactants, flavors, fragrances, pH modifiers, bulking agents, and mixtures thereof.

17. The method of claim 12, wherein the extract of Cassytha filiformis is provided in an amount of 1.2 grams to 1.8 grams per dose; or wherein the extract of Cuscuta sandwichiana is provided in an amount of 0.2 grams to 0.8 grams per dose; or wherein the Cassytha filiformis is provided in an amount of 1.2 grams to 1.8 grams per dose and the Cuscuta sandwichiana is provided in an amount of 0.2 grams to 0.8 grams per dose.

18. The method of claim 12, wherein the Cassytha filiformis, Cuscuta sandwichiana, or both, are provided at least 30 minutes before sleep.

19. The method of claim 12, further comprising the step of identifying a subject in need of improved sleep prior to providing the composition.

20. The method of claim 12, wherein a whole plant powder of Cassytha filiformis, Cuscuta sandwichiana, or both is provided to the subject.

21. The method of claim 12, wherein the composition is a whole plant powder of Cassytha filiformis, and the composition further comprises a source of Vitamin E.

22. The method of claim 12, further comprising the step of extracting the composition with water that is sufficiently hot to extract aporphine alkaloids, or Vitamin E, respectively, or boiling water.

23. The method of claim 12, wherein the composition consists essentially of between 2.0 grams to 3.0 grams per day of the water extract of Cassytha filiformis, Cuscuta sandwichiana, or both.

24. The method of claim 12, wherein the composition consists of between 2.0 grams to 3.0 grams per day of the water extract of Cassytha filiformis, Cuscuta sandwichiana, or both.

25. A method for evaluating the amount of an extract Cassytha filiformis, Cuscuta sandwichiana, or both, adapted for oral administration needed to induce or improve sleep, the method comprising:
determining an average amount of time needed to reach a state of sleep in one or more subjects;
providing an amount of the extract Cassytha filiformis, Cuscuta sandwichiana, or both, orally to a first subset of the subjects, and a placebo to a second subset of the subjects;
determining an average amount of time needed to reach a state of sleep in the first subset of the subjects, and the second subset of the subjects; and
determining if the amount of the extract Cassytha filiformis, Cuscuta sandwichiana, or both, induces sleep in a shorter time, improves a quality of sleep, or both that is statistically significant as compared to any reduction occurring in the second subset of patients, wherein a statistically significant reduction indicates that the candidate drug is useful in treating said disease state.

26. A composition consisting essentially of an extract of Cassytha filiformis, an extract of Cuscuta sandwichiana, or both, adapted for extraction with hot water and oral administration in an amount sufficient to induce or improve sleep.

27. The composition of claim 26, wherein both the extracts of Cassytha filiformis and Cuscuta sandwichiana are provided in an amount that is synergistic for induce sleep, improve sleep, or both; or wherein the extract of Cassytha filiformis is provided in an amount of 1.2 grams to 1.8 grams per dose; or wherein the extract of Cuscuta sandwichiana is provided in an amount of 0.2 grams to 0.8 grams per dose; or wherein the Cassytha filiformis is provided in an amount of 1.2 grams to 1.8 grams per dose and the Cuscuta sandwichiana is provided in an amount of 0.2 grams to 0.8 grams per dose.

28. The composition of claim 26, wherein the Cassytha filiformis, Cuscuta sandwichiana, or both, are provided at least 30 minutes before sleep.

29. A composition consisting of an extract of Cassytha filiformis, an extract of Cuscuta sandwichiana, or both, adapted for extraction with hot water and oral administration in an amount sufficient to induce sleep.

30. The composition of claim 29, wherein both the extracts of Cassytha filiformis and Cuscuta sandwichiana are provided in an amount that is synergistic to induce sleep, improve sleep, or both; or wherein the extract of Cassytha filiformis is provided in an amount of 1.2 grams to 1.8 grams per dose; or wherein the extract of Cuscuta sandwichiana is provided in an amount of 0.2 grams to 0.8 grams per dose; or wherein the Cassytha filiformis is provided in an amount of 1.2 grams to 1.8 grams per dose and the Cuscuta sandwichiana is provided in an amount of 0.2 grams to 0.8 grams per dose.

31. The composition of claim 29, wherein the Cassytha filiformis, Cuscuta sandwichiana, or both, are provided at least 30 minutes before sleep.

32. A method for inducing or improving sleep in a patient in need thereof comprising administering to the patient a plant composition, the plant composition consisting essentially of an extract of Cassytha filiformis, Cuscuta sandwichiana, or both, adapted for oral administration and in an amount sufficient to induce or improve sleep.

33. A method for inducing or improving sleep in a patient in need thereof comprising administering to the patient a plant composition, the plant composition consisting of an extract of Cassytha filiformis, Cuscuta sandwichiana, or both, adapted for oral administration and in an amount sufficient to induce or improve sleep.
